(19) United States
(12) Patent Application Publication
(10) Pub. No.: US 2005/0182474 A1
(43) Pub. Date: Aug. 18, 2005

(54) Coated stent having protruding crowns and elongated struts

(75) Inventors: Ryan Alexander Jones, Higley, AZ (US); John D. Kantor, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/927,305

(22) Filed: Aug. 26, 2004

Related U.S. Application Data
(60) Provisional application No. 60/544,550, filed on Feb. 13, 2004.

(51) Int. Cl. A61F 2/06

(52) U.S. Cl. 623/1.11

(57) ABSTRACT

The present invention provides a stent comprising a stent framework having a plurality of stent framework rings. At least one stent framework ring includes a plurality of interconnected crowns and struts with at least one protruding crown formed by two elongated struts. The protruding crowns of one stent framework ring are connected to corresponding crowns of an adjacent stent framework ring. A system for treating a vascular condition, a method of manufacturing a stent, and a method of reducing polymer bridging within a drug-polymer coated stent are also disclosed.

### RELATED APPLICATIONS

This application claims the benefit of U.S. Provisional Patent Application 60/544,550 filed Feb. 13, 2004.

### FIELD OF THE INVENTION

This invention relates generally to biomedical stents. More specifically, the invention relates to a stent having a stent framework with protruding crowns and elongated struts to prevent coating defects of drug-polymer coatings.

### BACKGROUND OF THE INVENTION

Implantable biomedical stents are often deployed in the human body to reinforce blood vessels or other vessels within the body as part of surgical procedures for enlarging and stabilizing body lumens. With generally open tubular structures of metallic or polymeric material, endovascular stents typically have apertured or lattice-like walls, and can be either balloon expandable or self-expanding. A stent is usually deployed by mounting the stent on a balloon portion of a balloon catheter, positioning the stent in a body lumen, and expanding the stent by inflating the balloon. The balloon is then deflated and removed, leaving the stent in place.

There is increasing evidence that stent design influences angiographic restenosis and clinical outcomes. An ideal stent possesses a low profile, good flexibility to navigate tortuous vessels, adequate radiopacity, low recoil, sufficient radial strength, and high scaffolding ability. Favorable clinical outcomes are influenced by the material composition of the stent and any surface coatings, as well as the stent geometry and thickness that affect the expansion of the stent and reduce the recoil of the stent. A desirable endovascular stent provides an ease of delivery and necessary structural characteristics for vascular support, as well as long-term biocompatibility, antithrombogenicity, and antiproliferative capabilities. Some of the latest stent designs include coatings from which one or more drug agents are eluted. Stents are being coated with protective materials such as polymers to improve biocompatibility and prevent corrosion and with bioactive agents to help reduce tissue inflammation, thrombosis and restenosis at the site being supported by the stent.

An exemplary coating material, such as a polymeric matrix and therapeutic compounds in a solvent, may be applied to a stent by dipping, spraying, paint, or brushing techniques, as is known in the art. With any of these application techniques, it can be difficult to avoid excessive webbing, pooling, and bridging of coatings between closely located struts of the stent. These problems are often exacerbated when thicker coatings of drug polymers are used.

Partial solutions to webbing and having excess coating material on stent struts are recognized by those skilled in the art of manufacturing stents. For example, a manual-dipping process step that blows excessive material off the open framework of a tubular stent is disclosed in “Coating” by Taylor et al., U.S. Pat. No. 6,214,115 issued Apr. 10, 2001. The process addresses the problems of inconsistent drying and blockage of openings. Another dipping process that addresses the issues of blockage and bridging between the stent struts is disclosed by Hossainy et al. in “Process for Coating Stents,” U.S. Pat. No. 6,153,252 issued Nov. 28, 2000. Flow or movement of the coating fluid through the openings in the perforated medical device is used to avoid the formation of blockages and bridges. The flow system may use a perforated manifold inserted in the stent to circulate the coating fluid, or may place the stent on a mandrel or in a small tube that is moved relative to the stent during the coating process.

Another proposed solution to the webbing and bridging employs a thread that removes coating material located within the openings of a stent, as disclosed in “Process for Coating a Surface of a Stent,” Jayaraman, U.S. Pat. No. 6,517,889 issued Feb. 11, 2003. Potential problems of bridging or webbing within the lattice framework of the stent, however, are not addressed.

Accordingly, what is needed is an improved stent design optimized for drug-polymer coatings that helps prevent undesirable bridging or webbing and other coating defects. Such a stent design should provide a surface for coatings that can be well adhered, and a flexibility that maintains mechanical integrity during the deployment of the stent. The improved stent should have a scaffolding to keep the vessel open, high radial strength to resist vessel recoil, and excellent deliverability in tortuous or challenging anatomy. Additionally, an associated system for treating a vascular condition, a method of manufacturing a stent, and a method of reducing polymer bridging within a drug-polymer coated stent are needed.

### SUMMARY OF THE INVENTION

One aspect of the invention provides a system for treating a vascular condition, which includes a catheter and a stent coupled to the catheter. The stent includes a stent framework having a plurality of stent framework rings. At least one stent framework ring includes a plurality of interconnected crowns and struts with at least one protruding crown formed by two elongated struts. The protruding crowns of one stent framework ring are connected to corresponding crowns of an adjacent stent framework ring.

Another aspect of the invention provides a stent comprising a stent framework having a plurality of stent framework rings. Each stent framework ring includes a plurality of interconnected crowns and struts with at least one protruding crown formed by two elongated struts. The protruding crowns of one stent framework ring are connected to corresponding crowns of an adjacent stent framework ring.

Another aspect of the invention is a method of manufacturing a stent. A plurality of stent framework rings is provided. At least one stent framework ring includes a plurality of interconnected crowns and struts with at least one protruding crown formed by two elongated struts. The protruding crowns of one stent framework ring are fastened to corresponding crowns of an adjacent stent framework ring, and a stent framework is formed.

Another aspect of the invention is a method of reducing polymer bridging within a drug-polymer coated stent. A plurality of stent framework rings are provided, wherein at least one stent framework ring includes a plurality of interconnected crowns and struts with at least one protruding crown formed by two elongated struts. The protruding crowns of one stent framework ring are fastened to corresponding crowns of an adjacent stent framework ring, and a stent framework is formed. A drug-polymer coating is applied onto the stent framework. Coated non-protruding crowns of adjacent stent framework rings remain separated after the drug-polymer coating is applied.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The foregoing aspects and other attendant advantages of the present invention will become more readily appreciated by the detailed description taken in conjunction with the accompanying drawings.

### BRIEF DESCRIPTION OF THE DRAWINGS

Various embodiments of the present invention are illustrated by the accompanying figures, wherein:

FIG. 1 is an illustration of a system for treating a vascular condition including a catheter and a stent coupled to the catheter, in accordance with one embodiment of the current invention;

FIG. 2 is an illustration of a stent framework having a plurality of stent framework rings without protruding crowns;

FIG. 3a is an illustration of a portion of a drug-polymer coated stent showing polymer bridging of the drug-polymer coating between crowns of adjacent stent framework rings;

FIG. 3b is an illustration of a portion of a drug-polymer coated stent with an expanded intersegmental distance between crowns of adjacent stent framework rings without polymer bridging of the drug-polymer coating, in accordance with one embodiment of the current invention;

FIG. 4 is an illustration of a stent framework having a plurality of stent framework rings that include at least one protruding crown on each ring segment, in accordance with one embodiment of the current invention;

FIG. 5 is an illustration of a stent framework having a plurality of stent framework rings including two end rings, in accordance with one embodiment of the current invention;

FIG. 6 is an illustration of a stent framework having a plurality of stent framework rings including two end rings, in accordance with another embodiment of the current invention; and

FIG. 7 is a flowchart of a method for reducing polymer bridging within a drug-polymer coated stent, in accordance with one embodiment of the current invention.

### DETAILED DESCRIPTION OF THE PRESENTLY PREFERRED EMBODIMENTS

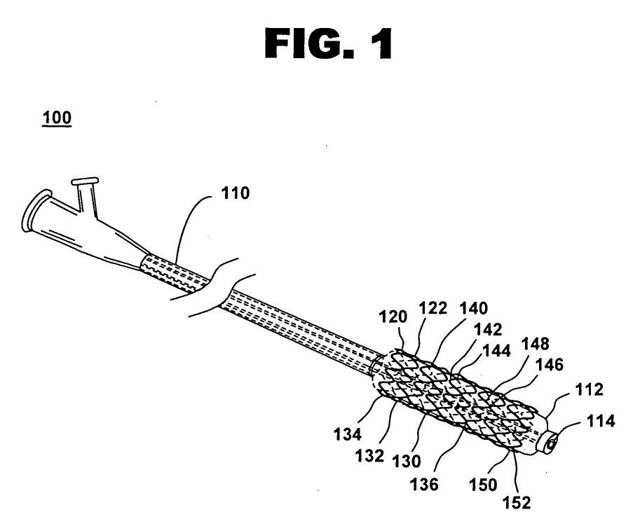

FIG. 1 is an illustration of a system for treating a vascular condition including a catheter and a stent, in accordance with one embodiment of the present invention at 100. Vascular condition treatment system 100 includes a catheter 110 and a stent 120 coupled to catheter 110. When deployed in the body, stent 120 provides support to vessel walls and effectively clears occlusions and other blockages in the region of deployment. To reduce the chance of restenosis or other medical conditions from occurring in the vicinity of the stent, stent 120 may include a drug-polymer coating 150 disposed on stent framework 122 of stent 120. To reduce the possibility of excess drug-polymer coating material between portions of stent framework 122, the intersegmental distance between unconnected crowns 142 of stent framework 122 is increased to separate crowns 142 and avoid the potential for polymer bridging of drug-polymer coating 150 between portions of stent framework 122 that are not directly connected, and to minimize cracking or flaking of drug-polymer coating 150 when stent 120 is deployed or otherwise flexed when in the body.

Stent 120 with or without drug-polymer coating 150 may be used, for example, as a cardiovascular stent, a peripheral stent, an abdominal aortic aneurysm stent, a cerebral stent, a carotid stent, or an endovascular stent. Insertion of stent 120 into a vessel of the body helps treat, for example, heart disease, various cardiovascular ailments, and other vascular conditions. Catheter-deployed stent 120 typically is used to treat one or more blockages, occlusions, stenoses, or diseased regions in the coronary artery, femoral artery, peripheral arteries, and other arteries in the body. Treatment of vascular conditions involves the prevention or correction of various ailments and deficiencies associated with the cardiovascular system, the cerebrovascular system, urinogenital systems, biliary conduits, abdominal passageways and other biological vessels within the body. Generally tubular in shape with flexibility to bend along a central axis, stent 120 expands with the help of a stent deployment balloon 112 or self-expands when released for a self-expanding version.

Catheter 110 of an exemplary embodiment of the present invention includes balloon 112 that expands and deploys stent 120 within a vessel of the body. Stent 120 is coupled to catheter 110, and may be deployed by pressurizing a balloon coupled to the stent and expanding stent 120 to a prescribed diameter. A flexible guidewire (not shown) traversing through a guidewire lumen 114 inside catheter 110 helps guide stent 120 to a treatment site, and once stent 120 is positioned, balloon 112 is inflated by pressurizing a fluid such as a contrast fluid that flows through a tube inside catheter 110 and into balloon 112. Stent 120 is expanded by balloon 112 until a desired diameter is reached, and then the contrast fluid is depressurized or pumped out, separating balloon 112 from deployed stent 120. Alternatively, catheter 110 may include a sheath that retracts to deploy a self-expanding version of stent 120.

Stent 120 includes a stent framework 122 having a plurality of stent framework rings 130. Stent framework rings 130 are sinusoidally shaped, continuously formed in a loop or ring with smooth, rounded corners referred to as crowns 142 at each bend, and substantially straight segments in between crowns 142 referred to as struts 144. As stent 120 is deployed, crowns 142 and struts 144 bend and straighten as the stent is enlarged diametrically, with minimal contraction extensionally.

Stent 120 may include a polymeric base or a metallic base including a base material such as stainless steel, nitinol, tantalum, MP35N alloy, platinum, titanium, a suitable biocompatible alloy, a suitable biocompatible material, and combinations thereof.

Stent framework ring 130 includes at least one ring segment 140 having a plurality of interconnected crowns 142 and struts 144. Crowns 142 and struts 144 have a nominally uniform radius and length, respectively. Additionally, each ring segment 140 has at least one protruding crown 146 extending proximally or distally beyond non-protruding crowns 142. Protruding crowns 146 are readily formed, for example, with elongated struts 148 connected to each side of protruding crown 146.

Each ring segment 140 may have a repeated pattern of sequentially connected crowns 142 and struts 144 with at least one protruding crown 146 formed by two elongated struts 148. Alternatively, each ring segment 140 of stent framework ring 130 may have a plurality of sequentially connected crowns 142 and struts 144 with at least one protruding crown 146 formed by two elongated struts 148, where one or more ring segments 140 are non-repeating.

Protruding crowns 146 of one stent framework ring 130 are connected to corresponding crowns 142 on an adjacent stent framework ring 130. The corresponding crowns 142 on the adjacent stent framework ring 130 may be protruding as well, though need not be. Protruding crowns 146 of stent framework ring 130 are connected to corresponding crowns 142 on an adjacent stent framework ring 130 with, for example, a welded joint 136. Alternatively, protruding crowns 146 of stent framework ring 130 may be connected to corresponding crowns 142 on an adjacent stent framework ring 130 with a molded joint 136, such as when stent 120 is formed from polymeric materials by a molding or casting process.

To provide stent framework 122 with additional rigidity at one or both ends of stent 120, additional joints 136 may be formed between corresponding crowns of an end ring 134 and an adjacent interior stent framework ring 132. Stent framework 122 may include one or two end rings 134 having a greater number of protruding crowns 146 than interior stent framework rings 132, with protruding crowns 146 of end rings 134 connected to corresponding non-protruding crowns 142 or protruding crowns 146 of adjacent interior stent framework rings 132.

Drug-polymer coating 150 may be disposed on stent framework 122 to provide desired therapeutic properties. An exemplary drug-polymer coating 150 comprises one or more therapeutic agents 152 that are eluted with controlled time delivery after the deployment of stent 120 within the body. Therapeutic agent 152 is capable of producing a beneficial effect against one or more conditions including coronary restenosis, cardiovascular restenosis, angiographic restenosis, arteriosclerosis, hyperplasia, and other diseases or conditions.

Drug-polymer coating 150 includes, for example, a therapeutic agent 152 such as rapamycin, a rapamycin derivative, a rapamycin analogue, an antirestenotic drug, an anti-cancer agent, an antisense agent, an antineoplastic agent, an antiproliferative agent, an antithrombogenic agent, an anticoagulant, an antiplatelet agent, an antibiotic, an anti-inflammatory agent, a steroid, a gene therapy agent, a therapeutic substance, an organic drug, a pharmaceutical compound, a recombinant DNA product, a recombinant RNA product, a collagen, a collagenic derivative, a protein, a protein analog, a saccharide, a saccharide derivative, a bioactive agent, a pharmaceutical drug, and combinations thereof.

Incorporation of a drug or other therapeutic agents 152 into drug-polymer coating 150 allows, for example, the rapid delivery of a pharmacologically active drug or bioactive agent within twenty-four hours following the deployment of stent 120, with a slower, steady delivery of a second bioactive agent over the next three to six months. The thickness of drug-polymer coating 150 may extend, for example, between 1.0 microns and 200 microns or greater in order to provide sufficient and satisfactory pharmacological benefit.

The intersegmental distance between adjacent stent framework rings 130 is therefore adapted to accommodate the thickness of drug-polymer coating 150 by extending selected struts 144 and forming protruding crowns 142 on segments of stent framework ring 130.

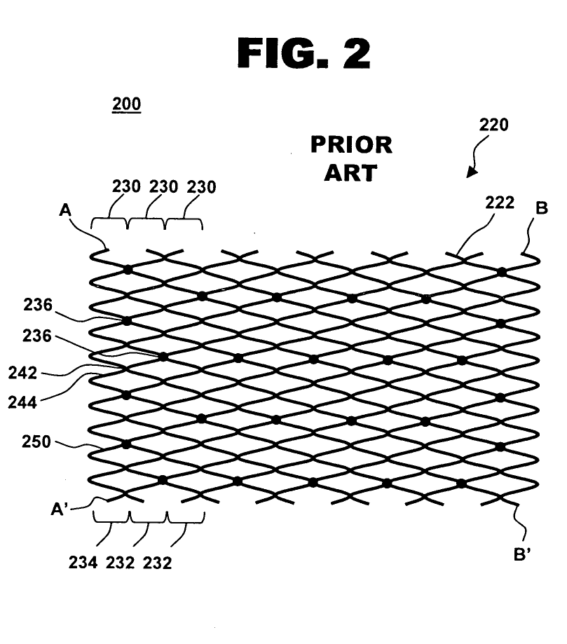

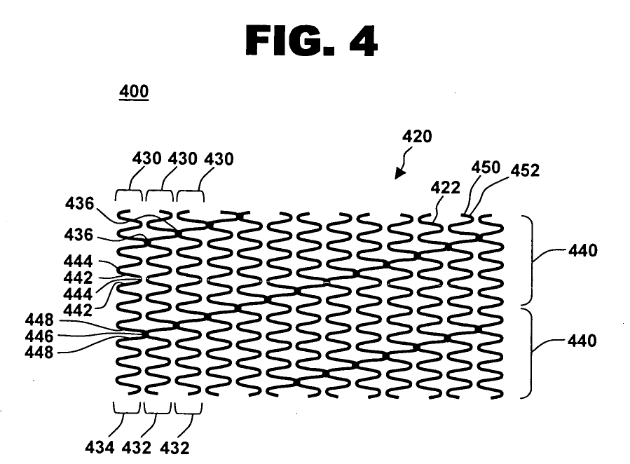

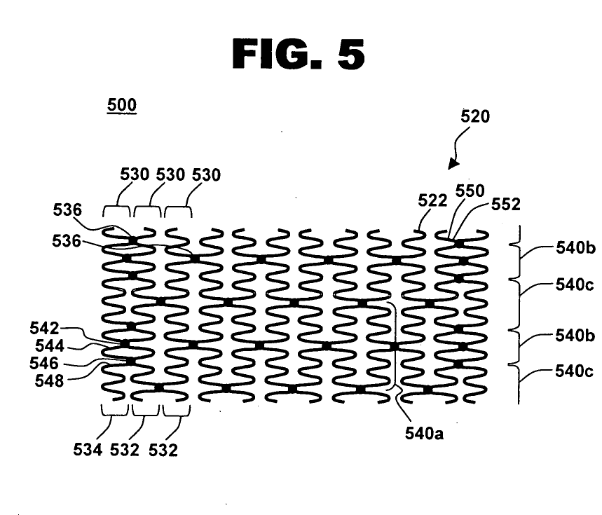

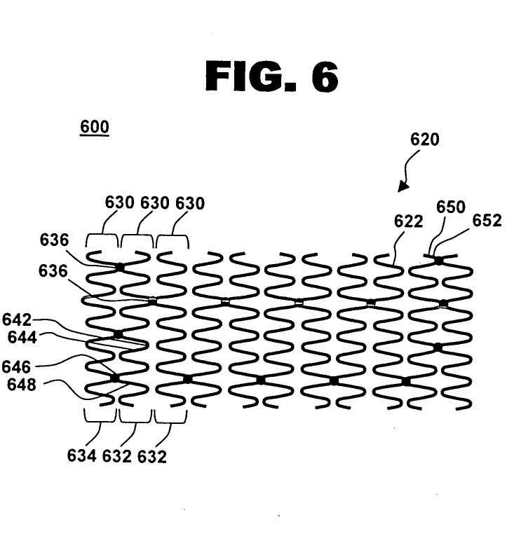

FIG. 2 is an illustration of a stent framework having a plurality of stent framework rings without any protruding crowns at 200. In this drawing and other similar drawings of FIG. 4, FIG. 5 and FIG. 6, the stent framework is shown unraveled, such that the stent is effectively cut along the length of one side, unrolled and flattened to clarify and illustrate salient characteristics of the invention. Point A is therefore connected continuously to point A′, point B is connected continuously to point B′, and other points on a line between A and B are connected continuously to corresponding points on a line between A′ and B′.

In this example of prior art, a stent 220 includes a stent framework 222 with a series of stent framework rings 230, each stent framework ring 230 having a plurality of crowns 242 and struts 244 of nominally uniform radii and length. Stent framework rings 230 are connected to adjacent stent framework rings 230 at one or more joints 236 where a crown 242 of one stent framework ring 230 is welded or otherwise connected to a corresponding crown 242 on an adjacent stent framework ring 230. Welded connections are spaced periodically to provide and control desired flexibility. An end ring 234 may be connected at numerous crowns 242 to an adjacent interior stent framework ring 232. Unconnected crowns 242 may occasionally touch or contact a corresponding crown 242 on adjacent stent framework ring 230 prior to expansion and even after expansion and deployment of stent 220. Contact between unconnected crowns 242 with a drug-polymer coating 250 may result in abrasion, cracking, or flaking of the coating in the vicinity of the contacting crowns. Improvements to the design can be made by increasing the intersegmental distance between adjacent, unconnected crowns 242 to decrease polymer bridging of drug-polymer coating 250 during its application and to reduce inadvertent contact between crowns 242 during handling and use.

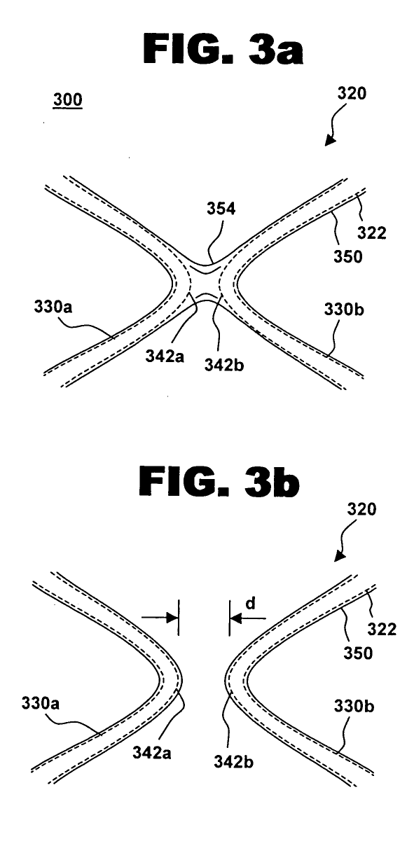

FIG. 3a is an illustration of a portion of a drug-polymer coated stent showing polymer bridging of the drug-polymer coating between crowns of adjacent stent framework rings at 300. Unconnected crowns 342a and 342b of adjacent stent framework rings 330a and 330b respectively have a polymeric bridge 354 of a drug-polymer coating 350 therebetween. When stent 320 with coated stent framework 322 is flexed or expanded, polymeric bridge 354 may inadvertently crack or flake off. In this case, the intersegmental distance between adjacent, unconnected crowns 342a and 342b is small or close to zero.

FIG. 3b is an illustration of a portion of a drug-polymer coated stent with an expanded intersegmental distance d between crowns of adjacent stent framework rings without polymer bridging of the drug-polymer coating, in accordance with one embodiment of the present invention. Unconnected crowns 342a and 342b of adjacent stent framework rings 330a and 330b respectively have no polymer bridging of drug-polymer coating 350. When stent 320 with coated stent framework 322 is flexed, coated crowns 342a and 342b do not contact each other and the coating is neither abraded nor cracked. In this case, the intersegmental distance is greater than zero and more than twice the thickness of drug-polymer coating 350.

FIG. 4 is an illustration of a stent framework having a plurality of stent framework rings with at least one protruding crown on each ring segment, in accordance with one embodiment of the present invention at 400. A stent 420 includes a stent framework 422 having a plurality of stent framework rings 430. Each stent framework ring 430 includes two ring segments 440 with a repeated pattern of sequentially connected crowns 442 and struts 444 with a right-protruding crown 446 and a left-protruding crown 446 formed by elongated struts 448. Protruding crowns 446 of stent framework ring 430 are connected to corresponding protruding crowns 446 on adjacent stent framework ring 430.

In this embodiment, each stent framework ring 430 has two protruding crowns 446 extending towards the right end or distal end of stent 420, and two protruding crowns 446 extending towards the left end or proximal end of stent 420, the distally protruding crowns 446 and the proximally protruding crowns 446 of each stent framework ring 430 interconnected by one elongated strut 448. Joints 436 connect protruding crowns 446 in a double-helically spiraling manner from the proximal end to the distal end of stent 420. Protruding crowns 446 of one stent framework ring 430 are connected to corresponding crowns 442 on adjacent stent framework ring 430 with, for example, a welded or a molded joint 436. It should be observed that in this embodiment, end rings 434 are the same as interior stent framework rings 432, such that a single ring-forming tool can be used to form all stent framework rings 430 for assembly into stent framework 422. End rings 434 have the same number of protruding crowns 446 as interior stent framework rings 432, with the same number of joints 436 as there are between adjacent interior stent framework rings 432.

Stent 420 may have a drug-polymer coating 450 with one or more therapeutic agents 452 disposed on stent framework 422. Coated non-protruding crowns 442 of adjacent stent framework rings 430 remain separated when drug-polymer coating 450 is disposed on stent framework 422.

FIG. 5 is an illustration of a stent framework having a plurality of stent framework rings including a pair of end rings, in accordance with one embodiment of the present invention at 500. A stent 520 includes a stent framework 522 having a plurality of stent framework rings 530 including interior stent framework rings 532 and end rings 534. Each interior stent framework ring 532 includes two ring segments 540a having a repeated pattern of sequentially connected crowns 542 and struts 544 with a right-protruding crown 546 and a left-protruding crown 546 formed by elongated struts 548 that are connected to protruding crowns 546. Protruding crowns 546 of stent framework ring 530 are connected to corresponding protruding crowns 546 on adjacent stent framework ring 530.

In this embodiment, each interior stent framework ring 532 has two protruding crowns 546 extending towards the right end or distal end of stent 520, and two protruding crowns 546 extending towards the left end or proximal end of stent 520. Distally protruding crowns 546 and proximally protruding crowns 546 of each interior stent framework ring 532 are interconnected by a set of three regular-length struts 544, two elongated struts 548, and four non-protruding crowns 542. Joints 536 are connected in a rotating manner with ninety-degree increments between adjacent interior stent framework rings 532. End rings 534 have minor differences from interior stent framework rings 532, though in this embodiment, the proximal and distal end rings 534 are identical to each other. End rings 534 have a greater number of protruding crowns 546 than interior stent framework rings 532, with an increased number of joints 536 between end rings 534 and adjacent interior stent framework rings 532. Protruding crowns 546 of one stent framework ring 530 are connected to corresponding crowns 542 on an adjacent stent framework 530 with, for example, welded or molded joint 536.

Stent 520 may have a drug-polymer coating 550 with one or more therapeutic agents 552 disposed on stent framework 522. Coated non-protruding crowns 542 of adjacent stent framework rings 530 remain separated when drug-polymer coating 550 is disposed on stent framework 522.

Each end ring 534 includes a set of interleaved ring segments 540b and 540c, with each ring segment including a plurality of sequentially connected crowns 542 and struts 544 having at least one protruding crown 546 formed by two elongated struts 548. Ring segment 540b extends, for example, from one protruding crown 546 to the next, with three non-protruding crowns 542, two non-elongated struts 544, and two elongated struts 548 in between. Ring segment 540c extends, for example, from one protruding crown 546 to the next, with five non-protruding crowns 542, four non-elongated struts 544, and two elongated struts 548 in between.

FIG. 6 is an illustration of a stent framework having a plurality of stent framework rings including an end ring, in accordance with another embodiment of the present invention at 600. A stent 620 includes a stent framework 622 having a plurality of stent framework rings 630 including interior stent framework rings 632 and end rings 634. Each interior stent framework ring 632 includes a repeated pattern of sequentially connected crowns 642 and struts 644 with a right-protruding crown 646 and a left-protruding crown 646 formed by elongated struts 648 connected to protruding crowns 646. From one protruding crown 646 to the next are six non-protruding crowns 642, five non-elongated struts 644, and two elongated struts 646. Protruding crowns 646 of stent framework ring 630 are connected to corresponding protruding crowns 646 on adjacent stent framework ring 630 with, for example, a welded or a molded joint 636.

Joints 636 are connected in a periodic manner with 180-degree increments between adjacent interior stent framework rings 632. End rings 634 have minor differences from interior stent framework rings 632, though the proximal and distal end rings 634 are identical to each other. End rings 634 have a greater number of protruding crowns 646 than interior stent framework rings 632, with an increased number of joints 636 between end rings 634 and adjacent interior stent framework rings 632.

Stent 620 may have a drug-polymer coating 650 with one or more therapeutic agents 652 disposed on stent framework 622. Coated non-protruding crowns 642 of adjacent stent framework rings 630 remain separated when drug-polymer coating 650 is disposed on stent framework 622.

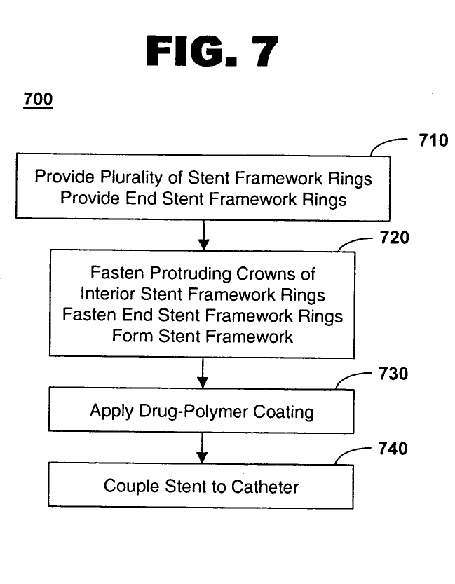

FIG. 7 is a flowchart of a method for manufacturing a stent and for reducing polymer bridging within a drug-polymer coated stent, in accordance with one embodiment of the present invention at 700.

A plurality of stent framework rings is provided, as seen at block 710. Each stent framework ring includes at least one ring segment having a plurality of interconnected crowns and struts with at least one protruding crown formed by two elongated struts. One or more end rings that have a greater number of protruding crowns than the interior stent framework rings may also be provided. The stent framework rings and end rings are formed, for example, with a loop or ring of wire or a stamped-out ring pattern from a sheet of metal that is positioned into a framework ring forming tool and compressed to form the non-protruding crowns and protruding crowns with the desired pattern and size. The initial stent material may include, for example, stainless steel, nitinol, tantalum, MP35N alloy, platinum, titanium, a suitable biocompatible alloy, a suitable biocompatible material, or combinations thereof. The stent framework rings are then cleaned using, for example, degreasers, solvents, surfactants, de-ionized water or other cleaners, as is known in the art.

The protruding crowns of one stent framework are fastened to corresponding crowns of an adjacent stent framework ring, as seen at block 720. For example, a set of stent framework rings and end rings are positioned on a mandrel and rotated to achieve the desired stent framework pattern. The protruding crowns of one stent framework ring are fastened to corresponding protruding or non-protruding crowns of the adjacent stent framework ring, for example, by forming a welded joint between the protruding crowns of one stent framework ring and corresponding crowns of the adjacent stent framework ring. Similarly, protruding crowns of the end rings are fastened to corresponding crowns of an adjacent interior stent framework ring.

The stent framework is formed, for example, by fastening the desired number of stent framework rings and end rings to each other to achieve the desired length of the stent. After the stent framework has been formed, the stent is cleaned and may be packaged and shipped for use, or it may be coated further with a drug-polymer or another coating before being packaged and delivered.

In an alternative embodiment, the stent framework is formed from metal or polymers with a cast or a mold, the cast or mold having molded joints between connected crowns and an enlarged intersegmental distance between unconnected crowns to reduce or eliminate polymeric bridges. In another embodiment, the stent framework is cut from small-diameter tubing with a laser or water jet cutting tool.

An optional drug-polymer coating is applied onto the stent framework, as seen at block 730. An exemplary drug polymer that includes a polymeric matrix and one or more therapeutic compounds is mixed with a suitable solvent to form a polymeric solution, and is applied using an application technique such as dipping, spraying, paint, or brushing. During the coating operation, the drug-polymer adheres to the stent framework and any excess drug-polymer solution may be removed, for example, by being blown off. In order to eliminate or remove any volatile components, the polymeric solution may be dried at room temperature or at elevated temperatures under dry nitrogen or another suitable environment. A second dipping and drying step may be used to increase the thickness of the drug-polymer coating, the thickness ranging between 1.0 microns and 200 microns or greater in order to provide sufficient and satisfactory pharmacological benefit.

The drug-polymer coating may be treated, for example, by heating the drug-polymer coating to a predetermined temperature to drive off any remaining solvent or to effect any additional crosslinking or polymerization. The drug-polymer coating may be treated with air drying or low-temperature heating in an air, nitrogen, or other controlled environment.

The drug-polymer coating may be applied before or after rolling the stent framework down to a desired diameter before insertion into the body. Coated non-protruding crowns of adjacent stent framework rings remain separated after the drug-polymer coating has been applied.

The coated or uncoated stent may be integrated into a system for treating vascular conditions such as heart disease by coupling the stent to the catheter, as seen at block 740. Exemplary finished stents are reduced in diameter, placed into the distal end of the catheter, and formed, for example, with an interference fit that secures the stent onto the catheter. Radiopaque markers may be attached to the stent or catheter to aid in the placement of the stent within the body. The catheter along with the drug-coated or non-coated stent may be sterilized and placed in a catheter package prior to shipping and storing. Additional sterilization using conventional medical means occurs before clinical use.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A system for treating a vascular condition, comprising:
a catheter; and
a stent coupled to the catheter, the stent including a stent framework having a plurality of stent framework rings, wherein at least one stent framework ring includes a plurality of interconnected crowns and struts with at least one protruding crown formed by two elongated struts and at least one non-protruding crown, wherein the protruding crowns of the at least one stent framework ring are connected to corresponding crowns of an adjacent stent framework ring, and wherein the stent framework has a drug-polymer coating disposed thereon, wherein coated non-protruding crowns of adjacent stent framework rings remain separated when the drug-polymer coating is disposed on the stent framework.

2. The system of claim 1 wherein the catheter includes a balloon used to expand the stent.

3. The system of claim 1 wherein the catheter includes a sheath that retracts to allow expansion of the stent.

4. The system of claim 1 wherein the stent framework comprises one of a metallic base or a polymeric base.

5. The system of claim 4 wherein the metallic base is selected from the group consisting of stainless steel, nitinol, tantalum, MP35N alloy, platinum, titanium, a suitable biocompatible alloy, a suitable biocompatible material, and a combination thereof.

6. The system of claim 1 wherein the at least one stent framework ring includes a plurality of ring segments, each ring segment having a repeated pattern of sequentially connected crowns and struts with at least one protruding crown formed by two elongated struts.

7. The system of claim 1 wherein the at least one stent framework ring includes a plurality of ring segments, each ring segment having a plurality of sequentially connected crowns and struts with at least one protruding crown formed by two elongated struts, wherein at least one ring segment of the plurality of ring segments is non-repeating.

8. The system of claim 1 wherein the plurality of stent framework rings includes an end ring having a greater number of protruding crowns than an interior stent framework ring.

9. The system of claim 1 wherein the protruding crowns of the at least one stent framework ring are connected to corresponding crowns of the adjacent stent framework ring with a welded joint.

10. The system of claim 1 wherein the protruding crowns of the at least one stent framework ring are connected to corresponding crowns of the adjacent stent framework ring with a molded joint.

11. The system of claim 1 wherein the stent is selected from the group consisting of a cardiovascular stent, a peripheral stent, an abdominal aortic aneurysm stent, a cerebral stent, a carotid stent, and an endovascular stent.

12. A stent comprising:
a stent framework having a plurality of stent framework rings, wherein at least one stent framework ring includes a plurality of interconnected crowns and struts with at least one protruding crown formed by two elongated struts and at least one non-protruding crown, wherein the protruding crowns of the at least one stent framework ring are connected to corresponding crowns of an adjacent stent framework ring, and wherein the stent framework has a drug-polymer coating disposed thereon, wherein coated non-protruding crowns of adjacent stent framework rings remain separated when the drug-polymer coating is disposed on the stent framework.

13. The stent of claim 12 wherein the stent framework comprises one of a metallic base or a polymeric base.

14. The stent of claim 13 wherein the metallic base is selected from the group consisting of stainless steel, nitinol, tantalum, MP35N alloy, platinum, titanium, a suitable biocompatible alloy, a suitable biocompatible material, and a combination thereof.

15. The stent of claim 12 wherein the stent is selected from the group consisting of a cardiovascular stent, a peripheral stent, an abdominal aortic aneurysm stent, a cerebral stent, a carotid stent, and an endovascular stent.

16. A method of manufacturing a stent, comprising:
providing a plurality of stent framework rings, wherein at least one stent framework ring includes a plurality of interconnected crowns and struts with at least one protruding crown formed by two elongated struts and at least one non-protruding crown;
fastening the protruding crowns of the at least one stent framework ring to corresponding crowns of an adjacent stent framework ring;
forming a stent framework; and
applying a drug-polymer coating onto the stent framework, wherein coated non-protruding crowns of adjacent stent framework rings remain separated after the drug-polymer coating is applied.

17. The method of claim 16 wherein fastening the protruding crowns of the at least one stent framework ring to corresponding crowns of the adjacent stent framework ring comprises forming a welded joint between the protruding crowns of the at least one stent framework ring and corresponding crowns of the adjacent stent framework ring.

18. The method of claim 16 further comprising:
providing an end ring having a greater number of protruding crowns than an interior stent framework ring; and
fastening the protruding crowns of the end ring to corresponding crowns of an adjacent interior stent framework ring.

19. A method of reducing polymer bridging within a drug-polymer coated stent, comprising:
providing a plurality of stent framework rings, wherein at least one stent framework ring includes a plurality of interconnected crowns and struts with at least one protruding crown formed by two elongated struts and at least one non-protruding crown;
fastening the protruding crowns of the at least one stent framework ring to corresponding crowns of an adjacent stent framework ring;
forming a stent framework; and
applying a drug-polymer coating onto the stent framework, wherein coated non-protruding crowns of adjacent stent framework rings remain separated after the drug-polymer coating is applied.
